(19) United States
(12) Patent Application Publication
(10) Pub. No.: US 2007/0026024 A1
(43) Pub. Date: Feb. 1, 2007

(54) Compositions and methods for emulsifying a pefluorocarbon with an oxygen-carrying surfactant

(76) Inventor: Thomas C. Drees, Pasadena, CA (US)

Correspondence Address: KUNZLER & ASSOCIATES, 8 EAST BROADWAY, SUITE 600, SALT LAKE CITY, UT, 84111, US

(21) Appl. No.: 11/190,433

(22) Filed: Jul. 27, 2005

(51) Int. Cl. A61K 9/00; A61K 33/00

(52) U.S. Cl. 424/400; 424/600

(57) ABSTRACT

A physiologically acceptable perfluorocarbon emulsion composition that includes perfluorodecalin and an oxygen-carrying fluorinated surfactant forming a stable emulsion in a continuous aqueous phase. The oxygen-carrying fluorinated surfactant may be fractionated to increase its physiological compatibility, and may further include a fatty acid radical perfluorinated to increase its oxygen-carrying capacity. The perfluorocarbon emulsion composition of the present invention thus exhibits improved stability and efficiency, broadening its application and effectiveness as an artificial oxygen carrier.

### BACKGROUND OF THE INVENTION

1. Field of the Invention

This invention relates to perfluorocarbon emulsions and more particularly relates to compositions and methods for emulsifying a perfluorocarbon with an oxygen-carrying surfactant to produce a physiologically acceptable intravascular oxygen carrier.

2. Description of the Related Art

The demand for blood is ever-increasing in America as it is worldwide, with baby boomers approaching retirement age and foreign conflict resulting in unavoidable casualties. Accident and burn victims, cancer patients, and other patients undergoing surgeries and medical treatments also require immense amounts of blood and blood products on a daily basis. In fact, one in twenty Americans will require a blood transfusion at some point in their lives.

The incredible and unceasing demand for blood, combined with serious shortages in the donor blood supply, has made achievement of a physiologically acceptable synthetic blood product a worthy goal of biomedical research, especially in recent years. Perfluorocarbons are chemically inert, synthetic molecules consisting primarily of carbon and fluorine atoms that form a colorless liquid. Because of their ability to physically dissolve significant quantities of gases, including oxygen and carbon dioxide, perfluorocarbons seem a logical substitute for blood. Despite such affable properties however, perfluorocarbons are hydrophobic, and thus not miscible with water. Accordingly, perfluorocarbons must be emulsified prior to intravenous use.

During the Vietnam War, the military vigorously pursued development of a hemoglobin-based blood substitute for battlefield use. During this same period of time, Dr. Leland Clark of Cincinnati Children's Hospital first began experimenting with perfluorocarbons as an alternative synthetic blood product. While the military was not immediately successful in developing a clinically acceptable hemoglobin-based blood substitute, initial work by Dr. Clark, Robert Geyer, Henry Sloviter, and others led to the production of Fluosol DA by the Green Cross Corporation of Japan, a first-generation purely synthetic oxygen carrier that showed considerable promise for human use.

Fluosol DA, however, was problematic in that the emulsion of perfluorocarbons in an aqueous phase was inherently unstable, both thermodynamically and kinetically. This instability required storage of the emulsion in a frozen state, and further required a laborious and time-consuming process of blending the emulsion with other ancillary solutions immediately before use. Further, sufficient oxygen supply and exchange required maintaining a patient on 70-100% oxygen during treatment with Fluosol DA.

Second generation synthetic oxygen carriers have improved upon Fluosol DA by utilizing smaller chain perfluorocarbon molecules to more effectively emulsify the perfluorocarbons, thereby allowing higher concentrations of active agent in the emulsion and thus higher oxygen-carrying capability. Second generation emulsions are also more stable than Fluosol DA, enabling storage at 4° C. for several months without significant degradation of activity.

Despite these improvements, manufacturing and stabilizing synthetic oxygen carriers remain great technological challenges as only droplets of around 0.16 μm or less in diameter are well-tolerated in physiological systems. Further, perfluorocarbon-based emulsions are immiscible, and therefore inherently unstable, in water. Known emulsification agents such as egg yolk phospholipids and lecithin also include extraneous components that threaten the stability of a final product useful as an intravenous oxygen carrier.

Accordingly, a need exists for compositions and methods for emulsifying a perfluorocarbon with an oxygen-containing surfactant to produce a physiologically acceptable artificial oxygen carrier. Beneficially, such compositions and methods would produce a fine perfluorocarbon emulsion having small particle diameter, increased affinity between perfluorocarbons and both water and perfluorocarbon phases of the emulsion, and increased oxygen carrying capacity. Such compositions and methods are disclosed and claimed herein.

### SUMMARY OF THE INVENTION

The present invention has been developed in response to the present state of the art, and in particular, in response to the problems and needs in the art that have not yet been fully solved by currently available compositions and methods for emulsifying a perfluorocarbon with a surfactant to produce a physiologically acceptable artificial oxygen carrier. Accordingly, the present invention has been developed to provide compositions and methods for emulsifying a perfluorocarbon with an oxygen-carrying surfactant that overcome many or all of the above-discussed shortcomings in the art.

The perfluorocarbon emulsion composition in accordance with certain embodiments of the present invention includes perfluorodecalin and an oxygen-carrying fluorinated surfactant forming a stable emulsion of the perfluorodecalin in a continuous aqueous phase. The perfluorodecalin may be provided in an amount between about five and about eighty-five percent by weight of the composition, while the oxygen-carrying fluorinated surfactant may be provided in an amount between about five and about fifty percent by weight of the composition. The oxygen-carrying fluorinated surfactant may include a fatty acid having between six and twelve carbon atoms, and in some embodiments, and may be perfluorinated to increase its oxygen-carrying capacity.

In one embodiment, the perfluorocarbon emulsion composition includes soy lecithin as the oxygen-carrying fluorinated surfactant. In other embodiments, the oxygen-carrying fluorinated surfactant may include one of phosphatidyl choline, phosphatidyl inositol, and phosphatidylethanolamine, where each of the foregoing is derived from the soy lecithin.

A method for making the perfluorocarbon emulsion composition may include providing soy lecithin, substituting on to the soy lecithin a fatty acid radical, and fluorinating the fatty acid radical to produce an oxygen-carrying fluorinated surfactant. The method may then include emulsifying, within a continuous aqueous phase, the oxygen-carrying fluorinated surfactant and perfluorodecalin to produce a physiologically acceptable artificial oxygen carrier.

In one embodiment, the fatty acid radical substituted onto the soy lecithin may include a carbon chain having between about twelve and about twenty-two carbon atoms. In some embodiments, the fatty acid radical may be perfluorinated to increase its oxygen-carrying capacity.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are disclosed to provide a thorough understanding of embodiments of the present invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

As used in this specification, the term “perfluorocarbon” refers to a carbon-flourine compound characterized by a high gas-dissolving capacity, low viscosity, and chemical and biological inertness. The term “perflourinated” refers to an organic structure where each of the hydrogen atoms associated with a carbon atom is replaced by fluorine.

The present invention includes compositions and methods for emulsifying a perfluorocarbon with an oxygen-carrying surfactant to produce a synthetic oxygen carrier that meets criteria for use in physiological systems. Specifically, a synthetic oxygen carrier produced in accordance with certain embodiments of the present invention may form a stable, fine emulsion that is non-toxic, non-mutagenic, and compatible with blood and endothelial cells, having insignificant pharmacological, physiological, and biochemical activity, and being excreted unchanged or forming harmless metabolites in physiological systems.

Indications for compositions and methods in accordance with the present invention may include acute surgical blood loss, high-risk angioplasty, pancreas preservation, transportation of transplant tissue including islet cells, islet cell viability/pre-islet cell transplant for diabetes mellitus, enhancement of tumor radiosensitivity, retinal surgery, acute myocardial infarction, acute ischemic stroke, various shock syndromes, and/or any other indications known to those in the art.

Indeed, in addition to use in physiological systems as a blood substitute, compositions in accordance with the present invention may be particularly advantageous for preserving transplant tissue during transport. Islet transplants, for example, have the potential to normalize blood sugar levels and prevent complications associated with diabetes mellitus. The fragile nature of islet cells, however, means that a significant portion of them are prone to die during harvest, storage, transport, and subsequent transplantation. Accordingly, methods of islet preservation and recovery having high islet yield are critical to the ultimate success of an islet transplant procedure. Compositions in accordance with the present invention may be used as a preservative solution to preserve a single layer of stored islet cells and thus enhance islet yield by minimizing oxygen depletion.

Compositions in accordance with the present invention may include a perfluorocarbon comprising the active pharmaceutical component. In some embodiments, for example, the perfluorocarbon may comprise a perfluorinated cyclohydrocarbon. In one embodiment, the perfluorocarbon includes at least one of the cis- and trans-isomers of perfluorodecalin, an inorganic, well-characterized molecule having an empirical formula of C₁₀ F₁₈ and a molecular weight of 462.08. Perfluorodecalin, also known as octadecafluorodecahydronaphthalene, perflunafene, and/or perfluorodecahydronaphthalene, has a boiling point of 142° C., a melting point of −10 to 142° C., a flash point of 40° C., and a bulk density of 1.917 kg/l at 25° C. Although perfluorodecalin is not soluble in water, embodiments of the present invention utilizing perfluorodecalin as the active pharmaceutical component may evidence low viscosity and small particle size, thereby facilitating a fine, stable emulsion that appears to the naked eye to be a physically homogeneous solution. The perfluorodecalin or other perfluorocarbon may be purified for medical use.

In some embodiments, perfluorodecalin may comprise between about five and eighty-five percent (5-85%) of the emulsion by composition weight. In other embodiments, the composition may further include a second active pharmaceutical component such as, for example, a second perfluorinated cyclohydrocarbon, where the second perfluorinated cyclohydrocarbon is also present in an amount between about five and eighty-five percent (5-85%) by composition weight. In still other embodiments, the perfluorodecalin or other primary active pharmaceutical component may be displaced entirely or in part by a perfluorinated or highly fluorinated oxygen-carrying surfactant, as described in more detail below.

Indeed, in certain embodiments, the composition may include a surfactant having significant fluorine content and properties of water dispersability that may be purified for medical use. In some embodiments, the surfactant may exhibit a high oxygen-carrying capacity sufficient to enable its dual function as a surfactant as well as the active pharmaceutical component. The surfactant may be prepared from naturally occurring precursor materials such as lecithin, from a synthesized counterpart of lecithin-derived materials, or from any other material known to those in the art. In one embodiment, the surfactant comprises soy lecithin, such as Phospholipon 90®G. Soy lecithin is a complex mixture of phospholipids, glycolipids, triglycerides, sterols, and small quantities of fatty acids, carbohydrates, and sphingolipids. The primary phospholipid components of soy lecithin include phosphatidyl choline (13-18%), phosphatidylethanolamine (10-15%), phosphatidyl inositol ((10-15%), phosphatidic acid (5-12%).

Naturally occurring lecithin, including soy lecithin, may be modified from its natural state to reduce the presence of spurious additives to the emulsion which are counter-indicated for use as a synthetic oxygen carrier in physiological systems. The amount of surfactant included in the composition may vary according to concentrations of active pharmaceutical components and depending on the specific properties of the emulsion sought, although in most cases the surfactant may comprise between about five and eighty-five percent (5-85%) by composition weight.

In some embodiments, the surfactant may be reacted to form derivatives exhibiting greater compatibility with the water and perfluorocarbon phases of the emulsion. In one embodiment, the surfactant includes lecithin fractions modified for increased affinity with the perfluorocarbon and/or water phases of the emulsion. As mentioned above with particular reference to soy lecithin, lecithin fractions may include, for example, phosphatidyl choline, phophatidylethanolamine, inositol, choline, cephalin, and/or other lecithin fractions known to those in the art. Lecithin fractions may be modified by fluorination or by adding highly water dispersible ester radicals to the base molecule. In some embodiments, the lecithin fractions may comprise fluorinated phosphatidyl choline, phosphatidylethanolamine ester, and/or mixtures thereof.

In one embodiment, the surfactant is prepared by esterifying the lecithin fraction phosphatidyl choline with a fluorinated fatty acid glyceryl. Specifically, fluorinated fatty acid radicals may be substituted onto the choline at the glyceryl hydroxyls while leaving the phosphatidyl radical. Alternatively, the naturally occurring fatty acid components on phosphatidyl choline may be esterified with a fluorinated fatty alcohol to the same esters. In any case, the fatty acid or alcohol used for esterification may include between about six and eight carbon atoms.

An esterification reaction in accordance with certain embodiments of the present invention may be carried out by preparing a first solution including about ten percent (10%) by weight of a C₁₀ fluorinated or perfluorinated acid, including about ninety percent (90%) by weight of a C₂₀ perfluorinated solvent for the acid, and including about 0.1 percent (0.1%) by weight mineral acid such as hydrochloric or sulfuric acid. The first solution may be prepared by applying moderate heat of between about fifty and sixty degrees Centigrade (50-60° C.). A second solution may be prepared by saponifying phosphotidyl choline to glyceryl phosphatidyl choline. The esterification reaction may then be induced by slowly adding the second solution to the first solution at between about fifty and sixty degrees Centigrade (50-60° C.) to effect esterification. In other embodiments, an alcohol esterification process may be similarly performed, except that the step requiring removal of the acid groups of the choline prior to reacting with a fluorinated fatty alcohol may be omitted.

An emulsion including the fluorinated fatty acid esterified glyceryl phosphatidyl choline prepared above may be formed by adding a suitable amount of water to form an emulsion. The amount of water may range, for example, between about fifty and seventy percent (50-70%) by composition weight.

Certain embodiments of compositions in accordance with the present invention may further include inactive ingredients such as anticoagulants, preservatives, antioxidants and/or any other inactive ingredients known to those in the art to prevent composition degradation over time or to facilitate effective use of the composition in physiological systems. In one embodiment, for example, the composition includes the following active and inactive ingredients:

| Ingredient Name | g/47 g | % (w/w) | g/Liter |
|---|---|---|---|
| Perfluorodecalin | 29.9920 | 63.738 | 879.58 |
| Phospholipon 90G ® (Soy Lecithin) | 2.6980 | 5.734 | 79.12 |
| Glycine | 0.2993 | 0.636 | 8.78 |
| Disodium EDTA | 0.0061 | 0.013 | 0.18 |
| Sodium Phosphate, Monobasic, | 0.0184 | 0.039 | 0.54 |
| Monhydrate, Crystal | | | |
| Sodium Phosphate, Dibasic, Anhydrous | 0.0024 | 0.005 | 0.07 |
| Water | 14.0392 | 29.836 | 411.73 |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A perfluorocarbon emulsion composition in a physiologically acceptable continuous aqueous phase, the composition comprising:
perfluorodecalin; and
an oxygen-carrying fluorinated surfactant forming a stable emulsion of the perfluorodecalin in the continuous aqueous phase.

2. The perfluorocarbon emulsion composition of claim 1, wherein the perfluorodecalin is present in an amount between five and eighty-five percent by weight of the composition.

3. The perfluorocarbon emulsion composition of claim 1, wherein the oxygen-carrying fluorinated surfactant is present in an amount between fifty and seventy percent by weight of the composition.

4. The perfluorocarbon emulsion composition of claim 1, wherein the oxygen-carrying fluorinated surfactant comprises a fatty acid having between six and twelve carbon atoms.

5. The perfluorocarbon emulsion composition of claim 1, wherein the oxygen-carrying fluorinated surfactant is perfluorinated.

6. The perfluorocarbon emulsion composition of claim 1, wherein the oxygen-carrying fluorinated surfactant comprises soy lecithin.

7. The perfluorocarbon emulsion composition of claim 1, wherein the oxygen-carrying fluorinated surfactant comprises phosphatidyl choline.

8. The perfluorocarbon emulsion of claim 1, wherein the oxygen-carrying fluorinated surfactant comprises phosphatidyl inositol.

9. The perfluorocarbon emulsion composition of claim 1, wherein the oxygen-carrying fluorinated surfactant comprises phosphatidylethanolamine.

10. A perfluorocarbon emulsion composition for use as a physiologically acceptable artificial oxygen carrier, the composition comprising:
perfluorodecalin; and
a fluorinated soy lecithin forming a stable emulsion of the perfluorodecalin in a continuous aqueous phase.

11. The perfluorocarbon emulsion composition of claim 10, wherein the perfluorodecalin is present in an amount between five and eighty-five percent by weight of the composition.

12. The perfluorocarbon emulsion composition of claim 10, wherein the fluorinated soy lecithin is present in an amount between fifty and seventy percent by weight of the composition.

13. The perfluorocarbon emulsion composition of claim 10, wherein the fluorinated soy lecithin comprises phosphatidyl choline.

14. The perfluorocarbon emulsion composition of claim 13, wherein the phosphatidyl choline is perfluorinated.

15. The perfluorocarbon emulsion composition of claim 10, wherein the fluorinated soy lecithin comprises a fatty acid.

16. The perfluorocarbon emulsion composition of claim 15, wherein the fatty acid comprises a carbon chain having a length between twelve and twenty-two carbon atoms.

17. A method for making a perfluorocarbon emulsion composition adapted for use as a physiologically acceptable artificial oxygen carrier, the method comprising:
providing soy lecithin;
substituting onto the soy lecithin a fatty acid radical;
fluorinating the fatty acid radical to produce an oxygen-carrying fluorinated surfactant; and
emulsifying, within a continuous aqueous phase, the oxygen-carrying fluorinated surfactant and perfluorodecalin to produce a physiologically acceptable artificial oxygen carrier.

18. The method of claim 17, wherein the soy lecithin comprises one of phosphatidyl choline, phosphatidyl inositol, and phosphatidylethanolamine.

19. The method of claim 17, wherein substituting onto the soy lecithin a fatty acid radical comprises selecting a fatty acid radical having a carbon chain between twelve and twenty-two carbon atoms.

20. The method of claim 17, wherein fluorinating the fatty acid radical comprises perfluorinating the fatty acid radical.
